(19) United States
(12) Patent Application Publication
(10) Pub. No.: US 2026/0013919 A1
(43) Pub. Date: Jan. 15, 2026

(54) Resorptive Intramedullary Implant Between Two Bones or Two Bone Fragments

(71) Applicant: Stryker European Operations Holdings LLC, Portage, MI (US)

(72) Inventors: Bernard Prandi, Rennes (FR); Marc Augoyard, Tassin La Demi Lune (FR); Thomas Ledermann, Eschenbach (CH); Tristan Meusnier, Saint-Etienne (FR); Jacques Peyrot, Tassin La Demi Lune (FR); Judith Fellmann, Stafa (CH)

(21) Appl. No.: 19/333,538

(22) Filed: Sep. 19, 2025

Related U.S. Application Data
(63) Continuation of application No. 18/770,767, filed on Jul. 12, 2024.
Application No. 18/770,767 is a continuation of application No. 16/506,353, filed on Jul. 9, 2019, now Pat. No. 12,059,186.
Application No. 16/506,353 is a continuation of application No. 14/858,855, filed on Sep. 18, 2015, now Pat. No. 10,383,671.
Application No. 14/858,855 is a division of application No. 13/795,946, filed on Mar. 12, 2013, now Pat. No. 9,168,074.
Application No. 13/795,946 is a continuation of application No. 12/918,105, filed on Oct. 29, 2010, now Pat. No. 8,414,583.

(51) Int. Cl. A61B 17/86; A61B 17/00; A61B 17/16; A61B 17/68; A61B 17/72; A61B 17/88; A61F 2/30; A61F 2/42

(52) U.S. Cl. CPC A61B 17/8605; A61B 17/1655; A61B 17/68; A61B 17/7233; A61B 17/88; A61F 2/4225; A61F 2/4241; A61B 2017/00862; A61B 2017/681; A61B 17/7208; A61B 17/7225; A61B 17/7266; A61B 17/7283; A61B 2017/8655; A61F 2002/30062; A61F 2002/30563; A61F 2002/30622; A61F 2002/3085; A61F 2002/30878; A61F 2210/0004

(57) ABSTRACT

The invention relates to a resorptive intramedullary implant between two bones or two bone fragments. The implant includes a single-piece body (1) having a generally elongate shape and having, at each end, areas for anchoring to the bone portions in question, characterized in that one of said areas (A1) has a cylindrical cross-section while the other area (A2) has a flat cross-section.

### CROSS-REFERENCE TO RELATED APPLICATIONS

This application is a continuation of U.S. patent application Ser. No. 18/770,767, filed on Jul. 12, 2024, which is a continuation of U.S. Patent No. 16/506,353, filed on Jul. 9, 2019, now U.S. Pat. No. 12,059,186, which is a continuation of U.S. patent application Ser. No. 14/858,855, filed Sep. 18, 2015, now U.S. Pat. No. 10,383,671, which is a divisional of U.S. patent application Ser. No. 13/795,946, filed Mar. 12, 2013, now U.S. Pat. No. 9,168,074, which is a continuation of U.S. patent application Ser. No. 12/918,105, filed Oct. 29, 2010, now U.S. Pat. No. 8,414,583, which application is a U.S. national phase entry under 35 U.S.C. § 371 of International Application No. PCT/FR2009/051658, filed Sep. 2, 2009, published as WO 2010/029246, which claims priority from French Patent Application No. 0856035, filed Sep. 9, 2008, whose entire disclosures are herewith incorporated by reference.

### FIELD OF THE INVENTION

The invention relates to the technical field of orthopedic implants, particularly for arthrodesis and osteosynthesis.

More particularly, the invention relates to an intramedullary implant for arthrodesis between two bone parts or osteosynthesis between two bone fragments, particularly in the case of the hand or foot.

### BACKGROUND OF THE INVENTION

Different solutions have been proposed to achieve these functions.

For example, a solution comes from the teaching of patent application FR 2,884,406 [US 2008/0177262], of which the applicant of the present application is also the applicant. This patent describes an intramedullary osteosynthesis device constituted of an elongated body whose ends constitute anchor zones cooperating with the bone parts to be immobilized. The anchor zones are shaped and made of a material selected to enable insertion into the bone parts, then to ensure an anchor in the bone parts by preventing any rotational movement by resisting traction and by maintaining a compression force.

Another solution also comes from patent application FR 07.02003 [US 2010/0131014], also from the same applicant. This document describes an implant in the form of two anchor zones connected by a central zone and whose general shape is substantially inscribed in a very elongated rectangle of X-shape, so as to form in the anchor zones two legs adapted to move apart by elastic or shape-memory effect.

From this design, different criteria have been established to make the implant easy to place and efficient in order to create a primary and secondary stability for the osteosynthesis or arthrodesis site.

However, these solutions are not adapted for the case of an implant made of resorptive material.

### BRIEF SUMMARY OF THE INVENTION

From this state of the art, the object that the invention proposes to attain is further improving the anchor and the stability of the implant as well as its adaptation to the morphology of the implantation site when the implant is made of resorptive material.

To solve such a problem, a resorptive intramedullary implant between two bones or two bone fragments has been designed and developed; it is constituted, in a known manner, of a single-piece body having a general elongated shape with, at each end, zones for anchoring to the bone parts being considered. According to the invention, one of the zones has a cylindrical shape, whereas the other zone is flat.

Advantageously, the implant is made of a resorptive material whose mechanical properties are determined to last the time necessary for the consolidation, so that the implant is resorbed after six months. For example, the implant is composed of lactic acid polymer or copolymer (PLA, PGA . . . ).

Considering the specific mechanical characteristics of resorptive materials, and to solve the given problem of improving anchor and stability, the cylindrical cross-section is threaded and tapers in the direction of its free end.

To solve the given problem of enabling a deformation by elasticity, thus causing an expansion adapted to the geometry of the site and to the properties of the material, the flat cross-section zone has, substantially in its median portion, an opening adapted to enable elastic deformation of the zone. The opening defines at least two anchor arms.

It therefore appears that the combination of a cylindrical and threaded anchor zone and a flat-sectioned anchor zone is particularly advantageous considering the problem to be solved.

To solve the given problem of resisting the shear and flexion forces susceptible of occurring in the area of the bone site, between the two anchor zones, the body has a central zone of transition adapted to resist the shear and flexion forces occurring in the area of the bone site and adapted to serve as an abutment.

From this basic design of the implant, the anchor zones are either coaxial or angularly offset by between about 1° and 30° and, advantageously, by 10°. The bend between the anchor zones is located so as to substantially correspond to an arthrodesis line of the bones being considered.

### BRIEF DESCRIPTION OF THE DRAWINGS

The invention is explained in more detail hereinafter with reference to the attached drawings, in which:

FIG. 1 is a perspective view of the implant;

FIG. 2 is a front view of the implant before insertion into the bone part in question;

FIG. 3 is a side view corresponding to FIG. 2;

FIG. 4 is a view like FIG. 2 showing the position of the anchor arms of the flat section after insertion;

FIG. 5 is a perspective view of another advantageous embodiment of the implant;

FIGS. 6 and 7 show the installation of the implant into two bone parts.

### DETAILED DESCRIPTION

The implant according to the invention has a one-piece body 1 of elongated shape and having a first proximal zone A1 and a second distal zone A2. The entire implant body is made of a resorptive material whose mechanical properties are determined for the implant to be resorbed in no less than about 6 months. In one embodiment, the implant is composed of lactic acid polymer or copolymer (PLA, PGA . . . ).

As will be described later in the description, the zones A1 and A2 have anchor formations for the respective bone parts. Taking into account the specific characteristics of the resorptive material and to attain the given object of anchor and stability, the zone A1 is of a cylindrical shape section whereas the other zone A2 is flat.

The zone A1 has a generally cylindrical outer surface 1a with a limited taper toward its free end. The surface 1a has a helical rib forming a screwthread 1a1.

The zone A2 is flat and has substantially in its center, an opening 1b adapted to enable elastic deformation of the zone A2. More particularly, the opening 1b defines at least two anchor arms 1c and 1d, each having at least one outwardly projecting tooth 1c1, 1d1.

Advantageously, between the two zones A1 and A2 the body 1 has a central zone C for transition adapted to resist shear and flexion forces that can occur at the end of a bone. By way of nonlimiting example, this median zone C can have a length of about 3.5 mm and a thickness of about 2 mm, for an overall implant length comprised between about 15 and 25 mm and a diameter of about 2 or 3 mm at the zone A1.

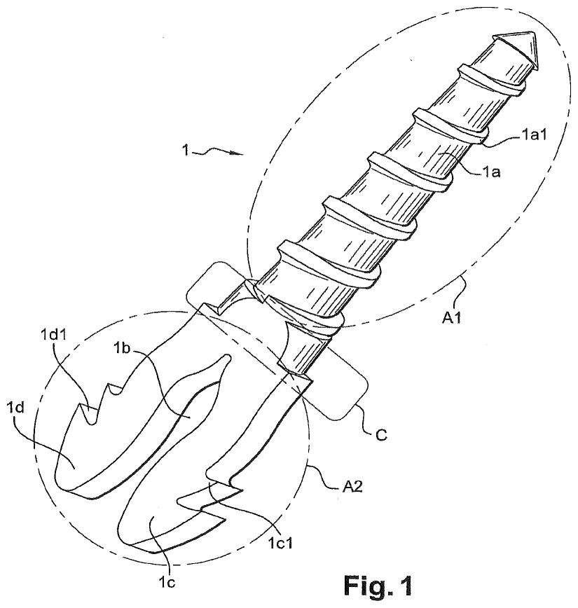

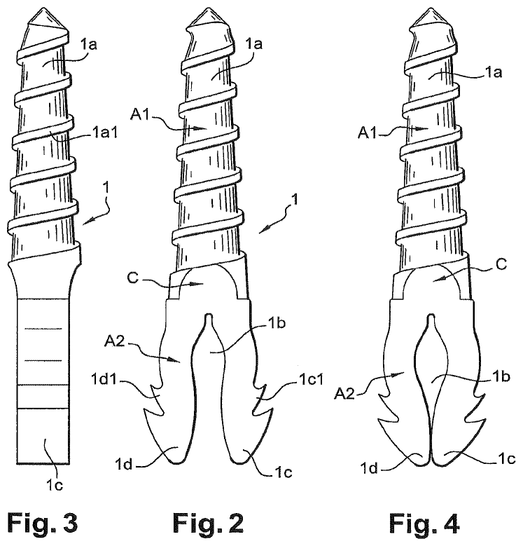

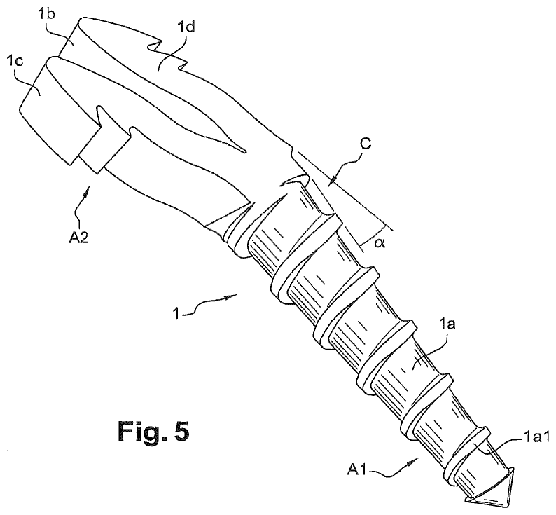

In the embodiment shown in FIG. 1, the two zones A1 and A2 are coaxial.

To solve the problem of adaptation to the shape of the implantation site, the anchor zones A1 and A2 can be offset at an angle α adapted to the geometry of the bone site. This angle α is comprised between about 1° and 30° and, advantageously, on the order of 10° when the implant is for foot arthrodesis (FIG. 5).

In this embodiment in which the two anchor zones are angularly offset, the bend is located so as to correspond substantially to the arthrodesis line of the bone parts being fused.

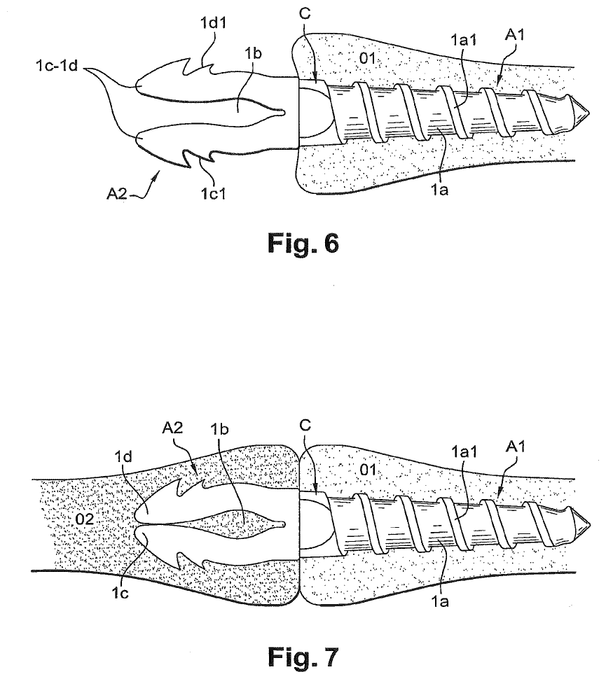

FIGS. 6 and 7 schematically show the positioning of the implant according to the invention between two bone parts O1 and O2. After suitable holes have been made in the bone by a rasp-type tool, the operator screws the thread 1a into the bone part O1 substantially up to the median zone C that serves as abutment preventing the implant from sinking too deeply into the bone (FIG. 6). The operator then fits the second bone part O2 back onto the anchor arms 1d and 1c of the zone A2, the anchor arms then spread and tighten by elasticity (FIG. 7).

The operative technique can be the following:
Drilling of the two holes with a conventional drill;
Preparation of the holes with a rasp for the flat side and a bone tap to form the inner screw thread on the cylindrical side;
Use of a screwdriver with a gripper end;
Screwing in the cylindrical side P1 [A1] for an arthrodesis IPP of the foot;
Fitting of the bone back onto the flat side [A2] of the implant.

The advantages are readily apparent from the description; in particular, it is to be emphasized and understood that the combination of the two anchor zones A1 and A2 of cylindrical and a flat shape, respectively, significantly enhances anchor and stability of the implant adapted to the geometry of the bone site and to the material properties, namely. a resorptive material.

1. (canceled)

2. An intramedullary implant for use between first and second bone parts, the implant being a monolithic body comprising:
a first threaded end for anchoring to the first bone part;
a central zone including a first surface extending along a first plane and a second surface extending along a second plane; and
a second end for anchoring to the second bone part, the second end having:
a first anchor arm having a third surface extending along a third plane, a fourth surface extending along a fourth plane, a first tooth, and a second tooth, the first and second teeth extending from the first anchor arm in a first direction,
a second anchor arm having a fifth surface extending along a fifth plane, a sixth surface extending along a sixth plane, a third tooth, and a fourth tooth, the third and fourth teeth extending from the second anchor arm in a second direction, the first and second directions being opposite one another, and
an opening between the first and second anchor arms to permit the first and second anchor arms to move with respect to each other,
wherein the first, third and fifth planes are parallel and the second, fourth, and sixth planes are parallel.

3. The intramedullary implant of claim 2, wherein the first, second, third, fourth, fifth and sixth planes are parallel.

4. The intramedullary implant of claim 2, wherein the intramedullary implant is made of a polymer.

5. The intramedullary implant of claim 2, wherein the first threaded end is connected to a first side of the central zone and the second end is connected to a second side of the central zone.

6. The intramedullary implant of claim 2, wherein the first tooth includes a first flat portion, the second tooth includes a second flat portion, the third tooth includes a third flat portion, and the fourth tooth includes a fourth flat portion.

7. The intramedullary implant of claim 6, wherein the first, second, third, and fourth flat portions are substantially coplanar.

8. The intramedullary implant of claim 2, wherein the first tooth includes first and second opposing surface portions, the second tooth includes third and fourth opposing surface portions, the third tooth includes fifth and sixth opposing surface portions, and the fourth tooth includes seventh and eighth opposing surface portions.

9. The intramedullary implant of claim 8, wherein each of the first, third, fifth, and seventh opposing surface portions define a first plane and each of the second, fourth, sixth, and eighth opposing surface portions define a second plane substantially parallel to the first plane.

10. The intramedullary implant of claim 2, wherein the first and second anchor arms are elastically deformable.

11. The intramedullary implant of claim 2, wherein the monolithic body includes a cannulation.

12. An intramedullary implant for use between first and second bone parts, the implant being a monolithic polymeric body comprising:
a first threaded end for anchoring to the first bone part;
a central zone including a first surface extending along a first plane and a second surface extending along a second plane;
a second end for anchoring to the second bone part, the second end having:
a first anchor arm having a third surface extending along a third plane, a fourth surface extending along a fourth plane, a first tooth, and a second tooth, the first and second teeth extending from the first anchor arm in a first direction,
a second anchor arm having a fifth surface extending along a fifth plane, a sixth surface extending along a sixth plane, a third tooth, and a fourth tooth, the third and fourth teeth extending from the second anchor arm in a second direction, the first and second directions being opposite one another, and
an opening between the first and second anchor arms to permit the first and second anchor arms to move with respect to each other, the first, third and fifth planes are parallel and the second, fourth, and sixth planes are parallel; and
a cannulation extending through the first threaded end, the central zone, and the second end.

13. The intramedullary implant of claim 12, wherein the first, second, third, fourth, fifth and sixth planes are parallel.

14. The intramedullary implant of claim 12, wherein the first threaded end is connected to a first side of the central zone and the second end is connected to a second side of the central zone.

15. The intramedullary implant of claim 12, wherein the first tooth includes a first flat portion, the second tooth includes a second flat portion, the third tooth includes a third flat portion, and the fourth tooth includes a fourth flat portion.

16. The intramedullary implant of claim 15, wherein the first, second, third, and fourth flat portions are substantially coplanar.

17. The intramedullary implant of claim 12, wherein the first tooth includes first and second opposing surface portions, the second tooth includes third and fourth opposing surface portions, the third tooth includes fifth and sixth opposing surface portions, and the fourth tooth includes seventh and eighth opposing surface portions.

18. The intramedullary implant of claim 17, wherein each of the first, third, fifth, and seventh opposing surface portions define a first plane and each of the second, fourth, sixth, and eighth opposing surface portions define a second plane substantially parallel to the first plane.

19. The intramedullary implant of claim 12, wherein the first and second anchor arms are elastically deformable.

20. The intramedullary implant of claim 19, wherein the cannulation is configured to accept a K-wire.

21. An implant for use between first and second bone parts, the implant being a monolithic polymeric body comprising:
a first threaded end for anchoring to the first bone part;
a central zone connected to the first threaded end and including a first surface extending along a first plane and a second surface extending along a second plane;
a second end connected to the central zone and for anchoring to the second bone part, the second end having:
a first anchor arm having a third surface extending along a third plane, a fourth surface extending along a fourth plane, a first tooth, and a second tooth, the first and second teeth extending from the first anchor arm in a first direction,
a second anchor arm having a fifth surface extending along a fifth plane, a sixth surface extending along a sixth plane, a third tooth, and a fourth tooth, the third and fourth teeth extending from the second anchor arm in a second direction, the first and second directions being opposite one another, and
an opening between the first and second anchor arms to permit the first and second anchor arms to move with respect to each other, the first, third and fifth planes are parallel and the second, fourth, and sixth planes are parallel; and
a cannulation extending through the first threaded end, the central zone, and the second end, the cannulation configured to accept a K-wire.
